(19) United States
(12) Patent Application Publication
(10) Pub. No.: US 2016/0206458 A1
(43) Pub. Date: Jul. 21, 2016

(54) ATRAUMATIC GASTROINTESTINAL ANCHOR, AND DELIVERY AND RETRIEVAL SYSTEMS FOR SAME

(71) Applicant: GI Dynamics, Inc., Lexington, MA (US)

(72) Inventors: Ryan Hanlon, Hudson, NH (US); Andres Chamorro, III, Ashland, MA (US); James Loper, Wales, MA (US); John Panek, Peabody, MA (US); Kelly Smith, Chelmsford, MA (US); Richard A. Gambale, Tyngsboro, MA (US)

(21) Appl. No.: 14/996,606

(22) Filed: Jan. 15, 2016

Related U.S. Application Data
(60) Provisional application No. 62/104,533, filed on Jan. 16, 2015.
(60) Provisional application No. 62/161,475, filed on May 14, 2015.

(51) Int. Cl. A61F 5/00

(52) U.S. Cl. CPC A61F 5/0076

(57) ABSTRACT

A gastrointestinal implant device comprises a proximal element configured to reside in a stomach to resist distal migration; a distal element configured to reside in an intestine to resist proximal migration, and one or more tethers coupling the proximal element to the distal element. The distal element comprises a wave anchor and a surface roughness element on a surface of at least a portion of the wave anchor. Delivery systems and retrieval techniques are also provided.

### RELATED APPLICATIONS

This application claims the benefit of U.S. Provisional Application No. 62/104,533, filed on Jan. 16, 2015 and U.S. Provisional Application No. 62/161,475, filed on May 14, 2015. The entire teachings of the above applications are incorporated herein by reference.

### BACKGROUND

There is an ongoing need to improve the duration of time over which gastrointestinal implants may safely be anchored in the gastrointestinal tract without occlusion, migration or other malfunction or drawbacks, particularly for implants that extend over at least a portion of the intestines.

Examples of such implants include those having flexible (floppy) sleeves extending into the intestine such as presented in U.S. Pat. Nos. 7,025,791; 7,122,058; 7,476,256; 7,608,114; 7,706,973; 7,771,382; 7,815,589; 7,837,643; 8,057,420; and those having restrictive orifices as presented in U.S. Pat. No. 7,771,382. All of those patents are incorporated by reference in their entirety.

In one particular drawback encountered in a pig model, the gastrointestinal implant may cause a section of the intestines to slide into an adjacent part of the intestines, and potentially even into the stomach, thereby “telescoping,” in a phenomenon known as intussusception. There is therefore an ongoing need to provide gastrointestinal implants that avoid such drawbacks, and that are as atraumatic as possible for the gastrointestinal tract.

### SUMMARY OF THE INVENTION

In accordance with an embodiment of the invention, there is provided a gastrointestinal implant device. The device comprises a proximal element configured to reside in a stomach to resist distal migration; a distal element configured to reside in an intestine to resist proximal migration, the distal element comprising a wave anchor and a surface roughness element on a surface of at least a portion of the wave anchor; and one or more tethers coupling the proximal element to the distal element.

In further, related embodiments, the wave anchor can comprise a diameter of between about 40 mm and about 60 mm, such as between about 50 mm and about 60 mm. The wave anchor can comprise a length of between about 25 mm and about 60 mm. The wave anchor can comprise a wire of a diameter of between about 0.016 inches and about 0.040 inches, such as between about 0.032 inches and about 0.035 inches. The surface roughness element can comprise a plurality of spheres on at least one crown of the wave anchor. The surface roughness element can comprise a helical coil wrap around at least a portion of the wave anchor. The helical coil wrap can comprise a wire of a diameter of between about 0.008 inches and about 0.020 inches, and can comprise an outside diameter of between about 0.080 inches and about 0.250 inches across a helix of the helical coil wrap. The helical coil wrap can comprise a coil wrap spring pitch of between about 8 coils per inch and about 60 coils per inch. The distal element can comprise an atraumatic covering over at least a portion of the helical coil wrap. The atraumatic covering can comprise a flexible sleeve material, such as a fluoropolymer, for example, expanded polytetrafluoroethylene.

In further, related embodiments, in normal use of the implant device, a central longitudinal axis of the planar proximal element, can be perpendicular to a plane in which the planar proximal element lies, is substantially perpendicular to a central longitudinal axis of a lumen of a pyloric sphincter of the stomach. The proximal element can be between about 40 mm and about 100 mm in size in a first dimension, and between about 0.5 mm and about 15 mm in size in a second dimension, orthogonal to the first dimension. The proximal element can be between about 50 mm and about 100 mm in size in the first dimension, and between about 0.5 mm and about 5 mm in size in the second dimension. The proximal element can comprise a hoop of between about 40 mm and about 70 mm diameter and of between about 0.5 mm and about 5 mm in thickness. The proximal element can comprise a hoop of wire of a thickness of between about 0.032 inches and about 0.036 inches.

In other related embodiments, only a single tether can couple the proximal element to the distal element; or more than one tether can couple the proximal element to the distal element. The distal element can be configured to seal to tissue of the intestine, thereby channeling chyme from the stomach into the intestine. The proximal element can be planar, and can comprise a hoop. The tether can comprises a flexible or rigid tether. The device can comprise no tissue penetrating features. The device can further comprise an unsupported, thin-walled sleeve coupled to the distal element. The proximal element can be without a seal to the stomach. The distal element can comprise a restrictor, and can further comprise an unsupported, thin-walled sleeve coupled to the distal element. The distal element can comprise an anchor of about the same length as the duodenal bulb. At least one of the proximal element, the tether and the distal element can be covered in an atraumatic material.

In further, related embodiments, the tether can comprise a suture. The tether can comprise at least one suture, the at least one suture being coupled to at least one crown of the wave anchor of the distal element, and to the proximal element. The at least one suture can be coupled to a ring that is coupled to the proximal element. The least one suture can comprise a single suture that is looped through both the at least one crown and through the ring that is coupled to the proximal element. The at least one suture can further comprise a second suture that is coupled to at least one other crown of the wave anchor and through the ring that is coupled to the proximal element. The tether can comprise at least one suture crimped together at a location between the proximal element and the distal element. The device can further comprise an overtube covering at least a portion of the at least one suture. The tether can be between about 10 mm and about 50 mm in length, and between about 0.5 mm and about 5 mm in diameter, and between about 1 mm and about 2 mm in diameter. The distal element can comprises a plurality of spokes, and the tether can be coupled to the plurality of spokes. The distal element can be configured to change shape upon transmission of force to the distal element by the tether. The tether can be coupled to at least one crown of the wave anchor, which may be a distal crown of the wave anchor. The distal element can be configured to open radially outwards upon exertion of tension by the tether. The proximal element can be loosely coupled to the tether, thereby permitting the proximal element to rotate independently of the tether. The proximal element can be coupled to a ring to which the tether is coupled. At least one of the proximal element and the distal element can further comprise a removal drawstring.

In another embodiment according to the invention, there is provided a delivery system for placing a gastrointestinal implant device in a mammalian gastrointestinal tract. The delivery system comprises a container assembly storing at least a proximal end of a distal element of the gastrointestinal implant device, the proximal end of the distal element including an anchor, and a distal end of the distal element including a flexible sleeve, coupled to the anchor, at least a portion of the flexible sleeve being folded into the container assembly, the distal element configured to reside in an intestine to resist proximal migration of the gastrointestinal implant device when implanted. The container assembly comprises an opening through which one or more tethers of the gastrointestinal implant device extends to an exterior of the container assembly, the one or more tethers coupling the distal element of the gastrointestinal implant device to a proximal element of the gastrointestinal implant device that is configured to reside in a stomach to resist distal migration of the gastrointestinal implant device when implanted. A grasper catheter holds at least a portion of the proximal element of the gastrointestinal implant device in a collapsed state exterior to the container assembly prior to release of the proximal element in the stomach, and is configured to release the at least a portion of the proximal element from the collapsed state in the stomach. An inner extension draws a portion of the sleeve from the anchor and from the container assembly as the anchor is retained therein, the inner extension comprising an atraumatic tip. An anchor plunger displaces the anchor from the container assembly.

In further, related embodiments, the grasper catheter can comprise a grasper hook at a distal end of the grasper catheter. The distal element of the gastrointestinal implant device can comprise a self-expanding wave anchor, held in a collapsed state inside the container assembly prior to being displaced from the container assembly. The proximal element of the gastrointestinal implant device can comprise a hoop. The hoop can be between about 40 mm and about 70 mm in diameter and between about 0.5 mm and about 5 mm in thickness. The grasper catheter can grasp a suture that holds the hoop in the collapsed state prior to release of the proximal element in the stomach. The inner extension can comprise an inner catheter releasably secured to a distal end of the sleeve. The atraumatic tip can comprise a ball. The atraumatic tip can comprise a guidewire rail channel that runs within the interior of the atraumatic tip.

In another embodiment according to the invention, there is provided a method of placing a gastrointestinal implant device in a mammal. The method comprises directing a container assembly into a mammalian gastrointestinal tract, the container assembly storing at least a proximal end of a distal element of the gastrointestinal implant device, the proximal end of the distal element including an anchor, and a distal end of the distal element including a flexible sleeve, coupled to the anchor, at least a portion of the flexible sleeve being folded into the container assembly. While directing the container assembly into the mammalian gastrointestinal tract, a grasper catheter is also directed into the mammalian gastrointestinal tract, the grasper catheter holding at least a portion of a proximal element of the gastrointestinal implant device in a collapsed state exterior to the container assembly, the proximal element of the gastrointestinal implant device being coupled to the distal element of the gastrointestinal implant device by one or more tethers extending through an opening in the container assembly. The at least a portion of the proximal element is released from the collapsed state in a stomach of the gastrointestinal tract. At least a portion of the sleeve is removed from the container assembly in an intestine of the gastrointestinal tract by extending a portion of the sleeve from the anchor and from the container assembly to a location in the intestine of the gastrointestinal tract that is distal relative to the container assembly while the anchor is retained in the container assembly. The anchor is subsequently removed from the container assembly in the intestine, such that the one or more tethers extend through a pylorus of the gastrointestinal tract to couple the distal element of the gastrointestinal implant device in the intestine to the proximal element of the gastrointestinal implant device in the stomach.

In further, related embodiments, the method can comprise holding the at least a portion of the proximal element in the collapsed state using a grasper hook at a distal end of the grasper catheter. The proximal element of the gastrointestinal implant device can comprise a hoop, the method comprising grasping a suture with the grasper catheter to hold the hoop in the collapsed state. The sleeve can be extended by advancing an inner catheter having an atraumatic tip. The method can comprise advancing the atraumatic tip over a guidewire that runs through a guidewire rail channel within the interior of the atraumatic tip. The method can comprise releasing a distal end of the sleeve from being releasably secured to the inner catheter. The atraumatic tip can comprise a ball, the method comprising releasing the ball into the gastrointestinal tract. The anchor can comprise a self-expanding wave anchor, and removing the anchor from the container assembly can comprise displacing the wave anchor from the container assembly with an anchor plunger to permit the wave anchor to expand in the intestine. The wave anchor can be expanded in the duodenal bulb.

In another embodiment, there is provided a method of removing or repositioning a gastrointestinal implant device in a mammal. The method comprises: engaging a distal element of the gastrointestinal implant device, in an intestine of the mammal, with a removal catheter; collapsing at least a proximal portion of the distal element with the removal catheter; moving the distal element through a pylorus of the mammal into a stomach of the mammal, with the removal catheter; moving the distal element through an esophagus of the mammal, with the removal catheter, while towing a proximal element of the gastrointestinal implant device behind the distal element in the stomach, the proximal element being coupled to the distal element by one or more tethers of the gastrointestinal implant device; and moving the proximal element through the esophagus with the removal catheter, at least a portion of the proximal element being collapsed by being forced against the tissue of the esophagus as the proximal element is moved through the esophagus.

In further related embodiments, engaging the distal element can comprise grasping a drawstring of the gastrointestinal implant device with a grasper of the removal catheter. Collapsing the at least a proximal portion of the distal element can comprise radially collapsing the at least a proximal portion of the distal element with a drawstring of the gastrointestinal implant device. The method can comprise placing an overtube in the esophagus, the overtube forcing the proximal element to collapse as the proximal element is moved through the esophagus. The distal element can comprise a wave anchor, and the proximal element can comprise a hoop.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a sectional view of a portion of the digestive tract in a body.

FIG. 2 is a perspective view of a gastrointestinal implant device comprising an anchoring device in accordance with an embodiment of the invention.

FIG. 3 is a diagram of a gastrointestinal implant device in accordance with an embodiment of the invention, comprising a wave anchor around which is wrapped the helical coil wrap.

FIGS. 4A-4D are views of a portion of a distal element in an anchor in accordance with an embodiment of the invention. In particular, FIG. 4A is a side view showing a portion of the wave anchor and helical coil wrap wrapped around it; FIG. 4B is a side view showing the wave anchor with helical coil wrap; FIG. 4C is a top view of the wave anchor with helical coil wrap; and FIG. 4D is a trimetric view of the wave anchor with helical coil wrap.

FIG. 5 is a sectional view of a body showing the gastrointestinal implant device of FIG. 2 implanted in the digestive system.

FIG. 6 is an implant device of the invention having plural tethers.

FIG. 7 is an implant device of the invention in which the tether is coupled to spokes on the distal element.

FIG. 8A is a side view, and FIG. 8B is a top view, of a wire form used as the basis of a wave anchor for a distal element of a gastrointestinal implant device, in accordance with an embodiment of the invention.

FIG. 9A is a top view, and FIG. 9B is a side view, of a hoop that can be used for the proximal element of a gastrointestinal implant device, in accordance with an embodiment of the invention.

FIG. 10 is an exploded view of helical coil wraps being assembled onto the wave anchor wire form as part of assembling a distal element, in accordance with an embodiment of the invention.

FIGS. 11A-11C are top views of a catheter-based delivery system, and FIGS. 12A-12C are side views of the same, in accordance with an embodiment of the invention. In FIGS. 11A-11C, the figures are to be viewed as a left portion view (FIG. 11A), center portion view (FIG. 11B) and right portion view (FIG. 11C) of the catheter-based delivery system, from a top view; while in FIGS. 12A-12C, the figures are to be viewed as a left portion view (FIG. 12A), center portion view (FIG. 12B) and right portion view (FIG. 12C) of the catheter-based delivery system, in accordance with an embodiment of the invention.

FIG. 13 is a cross-sectional diagram showing the internal details of a container assembly with implant therein, in accordance with an embodiment of the invention.

FIG. 14A is a disassembled view, and FIG. 14B is an assembled view, of a gastrointestinal implant device in accordance with an embodiment of the invention, illustrating use of a tether that includes at least one suture.

FIGS. 15A, 15B and FIG. 16 are diagrams illustrating assembly of a tether using one or more sutures, in accordance with an embodiment of the invention. FIG. 15A shows assembly of a suture with a crimp for use in a tether; FIG. 15B shows a suture structure with crimp, coupled to the crowns of a gastrointestinal implant; and FIG. 16 shows a fully assembled gastrointestinal implant using the crimped suture structure; in accordance with an embodiment of the invention.

### DETAILED DESCRIPTION OF THE INVENTION

A description of example embodiments of the invention follows.

There is provided an anchor for a gastrointestinal implant device. The anchor spans the pylorus, and therefore is called a transpyloric anchor. It is an objective of certain embodiments to provide the same or similar functionality as is provided by existing anchoring techniques for gastrointestinal implant devices, while having fewer side effects (such as bleeding, discomfort, migration and/or infection), and while having few or no tissue penetrating features.

Among other things, certain embodiments provide a method and apparatus for the application of a barrier sleeve in the digestive tract to limit the contact of food products in specific parts of the digestive tract and to provide enhanced satiety to patients with morbid obesity, enabling them to reduce their food intake. The sleeve may also be used for other treatments such as Type-2 diabetes through hormone triggers.

In a relaxed state, the stomach becomes flat, and thus a lightweight planar proximal element, in an anchor according to an embodiment of the invention, is able to orient itself in a plane within the relaxed stomach to cause as little trauma as possible to the stomach.

In accordance with certain embodiments, components of a transpyloric anchor include: (i) a proximal element in the stomach, which prevents distal migration; (ii) a distal element in the intestines, which both prevents proximal migration and may provide a seal; and (iii) one or more tethers, which connects the proximal and distal element.

In accordance with an embodiment of the invention, the distal element of the transpyloric anchor comprises a wave anchor and a surface roughness element, such as a helical coil wrap, discussed further below, on at least a portion of the wave anchor. The anchor may provide advantages in gastrointestinal anchoring, such as by reducing trauma and decreasing the danger of erosion of the intestines, migration of the implant or other potential drawbacks.

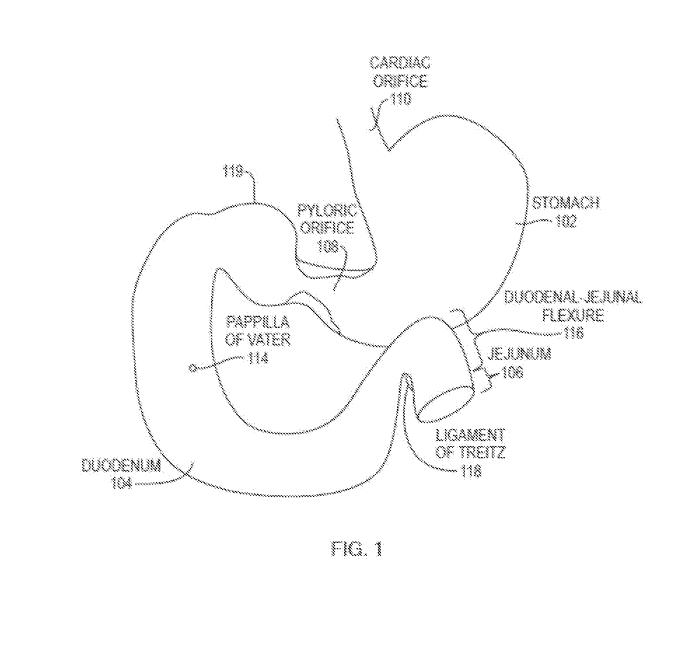

FIG. 1 is a sectional view of a portion of the digestive tract in a body. Food to be digested enters the stomach 102 through the cardiac orifice 110 from the esophagus. Chyme, a semi-fluid, homogeneous creamy or gruel-like material produced by gastric digestion in the stomach exits the stomach through the pyloric orifice (pylorus) 108 and enters the small intestine 112. The pylorus 108 is a distal aperture of the stomach 102 surrounded by a strong band of circular muscle. The small intestine, about nine feet in length, is a convoluted tube, extending from the pylorus 108 to the ileo-caecal valve where it terminates in the large intestine. The small intestine has three sections, the duodenum 104, jejunum 106 and the ileum (not shown). The first eight to ten inch section of the small intestine 112, the duodenum 104, is the shortest, widest and most fixed part of the small intestine 112.

The duodenum 104 has four sections: superior, descending, transverse and ascending which typically form a U-shape. The superior section is about two inches long and ends at the neck of the gall bladder. The superior section also defines a feature referred to as the duodenal bulb 119 that begins just distal to the pylorus 108 and extends for about 1 to 1.5 inches in an adult human. The duodenal bulb 119 defines a lumen therein that is slightly larger than the distal duodenum 104. Advantageously, the duodenal bulb 119 exhibits less motion than the pylorus 108 and even distal portions of the duodenum 104. Notably, the motion is substantially limited to contractions without having a significant linear component (i.e., no movement along the central axis of the intestine). However, the tissue thins as one moves away from the pylorus 108.

The descending section of the duodenum 104 is about three to four inches long and includes a nipple shaped structure (papilla of Vater) 114 through which pancreatic juice from the pancreas and bile produced by the liver and stored by the gall bladder enter the duodenum from the pancreatic and bile ducts. The pancreatic juice contains enzymes essential to protein digestion and the bile dissolves the products of fat digestion. The ascending section is about two inches long and forms the duodenal-jejunal flexure 116 where it joins the jejunum 106, the next section of the small intestine. The duodenal-jejunal flexure 116 is fixed to the ligament of Treitz 118 (musculus supensionus duodeni). The juices secreted in the duodenum break the partially digested food down into particles small enough to be absorbed by the body. The digestive system is described in Gray's Anatomy (“Anatomy of the Human Body,” by Henry Gray) and “Human Physiology,” Vander, 3^{rd} ed, McGraw Hill, 1980, the contents of which are incorporated herein by reference in their entirety.

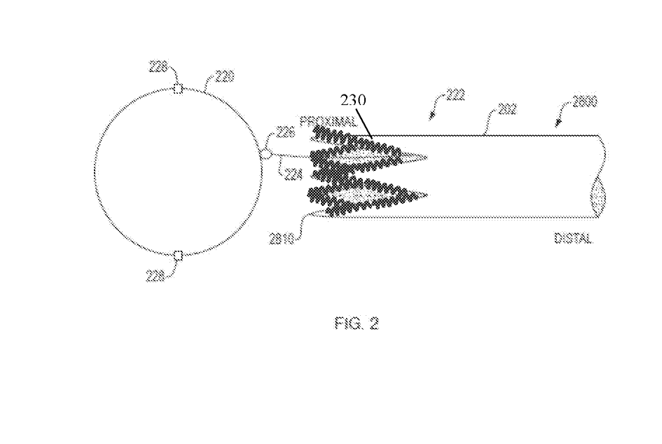

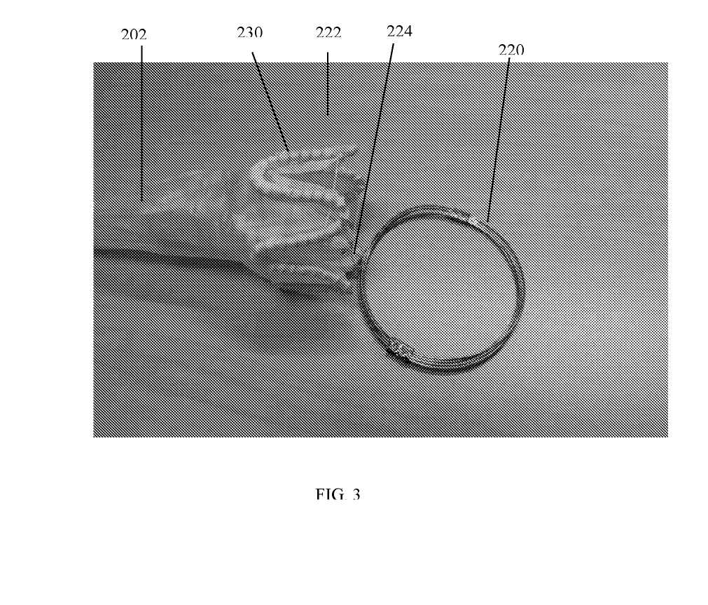

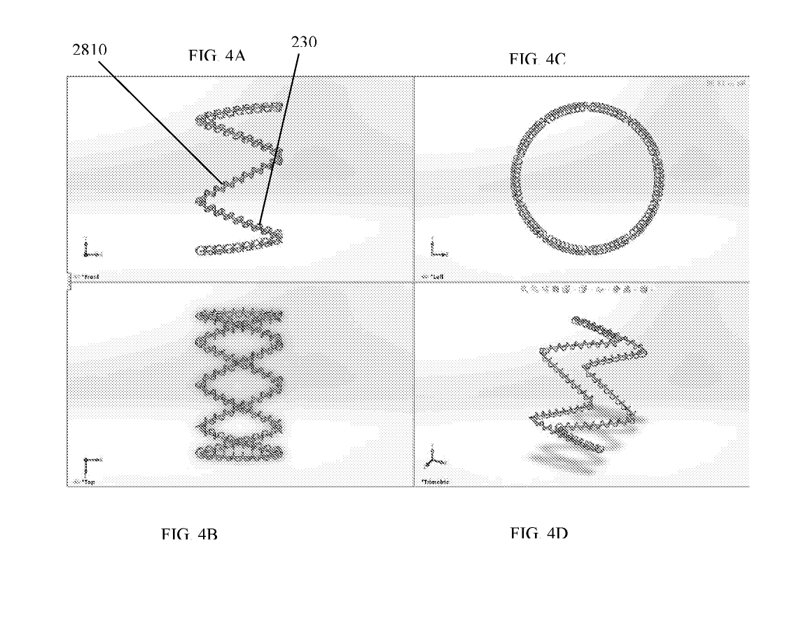

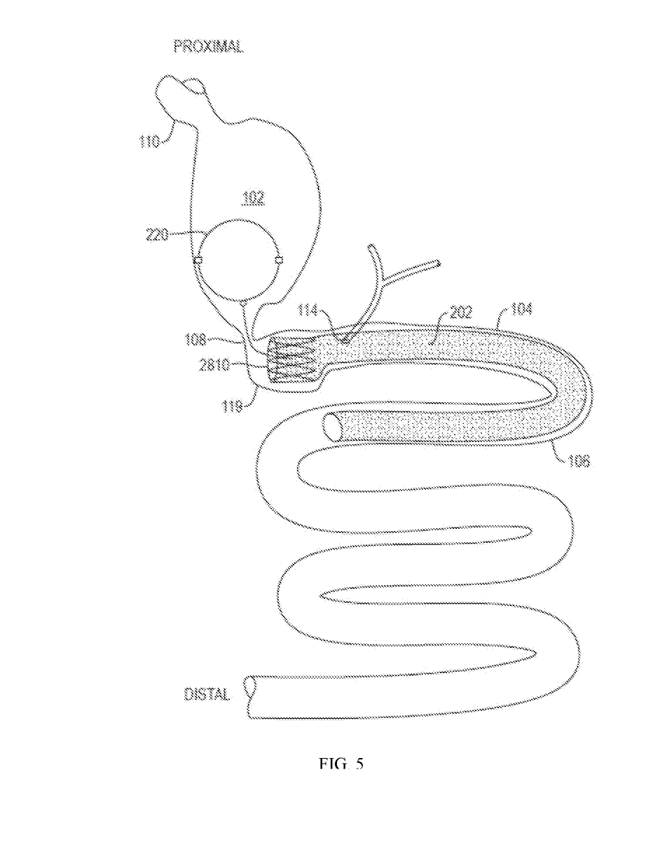

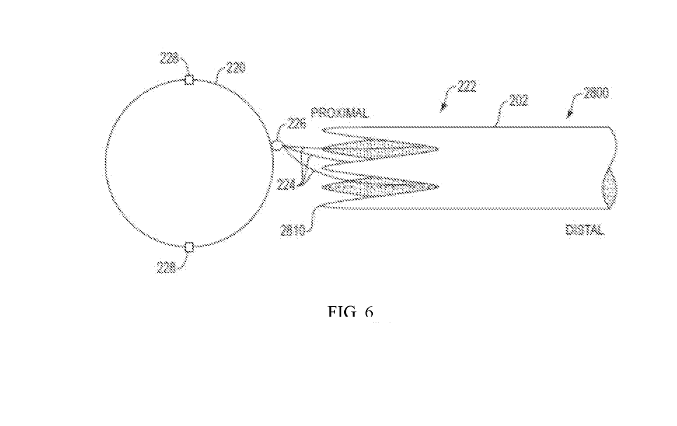

FIG. 2 is a perspective view of a gastrointestinal implant device 2800 comprising an anchoring device in accordance with an embodiment of the invention. The device 2800 comprises a proximal element 220, which may be planar, such as hoop, that is configured to reside in a stomach to resist distal migration; a distal element 222 configured to reside in an intestine to resist proximal migration and to provide a seal; and a single tether 224 coupling the planar proximal element 220 to the distal element 222. The distal element 222 comprises a wave anchor 2810 and a surface roughness element, such as a helical coil wrap 230 on at least a portion of the wave anchor 2810. Without being bound by theory, it is believed that the inclusion of such a surface roughness element, to increase the surface roughness of the wave anchor 2810, increases the stability of the anchor. Other types of surface roughness elements may be used, for example a plurality of spheres on one or more crowns of the wave anchor 2810. In one example, three 3/16 inch polypropylene spheres were used on each crown a wave anchor 2810, instead of the helical coil wrap 230 of FIG. 2. As shown in more detail in FIGS. 3 and 4A-4D, where the surface roughness element is a helical coil wrap 230, the helical coil wrap 230 can be wrapped around the wire that forms the wave anchor 2810, such that a central longitudinal axis of the helical coil wrap 230 extends substantially along the same route as the wire that forms the wave anchor, i.e., being helically wrapped around at least a portion of the path taken by the wire that forms the wave anchor.

FIG. 3 is a diagram of a gastrointestinal implant device 2800 in accordance with an embodiment of the invention, comprising a wave anchor 2810 around which is wrapped the helical coil wrap 230. The helical coil wrap 230 may be formed of collapsible wire, formed from a resilient metal such as a heat-treated spring steel, stainless steel, or from an alloy such as NiTi alloy commonly referred to as Nitinol. Other alloys include nickel-cobalt-chromium-molybdenum alloys possessing a unique combination of ultrahigh tensile strength, such as MP35N. Additionally, the helical coil wrap 230 can be formed from a polymer and/or a composite having similar properties. The helical coil wrap 230 can be manufactured from a single strand, such as a wire, contoured into the desired shape. Alternatively, the helical coil wrap 230 can be manufactured from multi-strands of the same or different materials similarly contoured to the desired shape. In some embodiments, the helical coil wrap 230 can be cut into the helical shape from tubular stock of the desired material, such as Nitinol. The helical coil wrap 230 may, for example, comprise a wire of a diameter of between about 0.008 inches and about 0.020 inches. The helical coil wrap may comprise an outside diameter of between about 0.080 inches and about 0.250 inches across the helix of the helical coil wrap. Further, the helical coil wrap may comprise a coil wrap spring pitch of between about 8 coils per inch and about 60 coils per inch. An atraumatic covering may cover at least a portion of the helical coil wrap. For example, the atraumatic covering may comprise a flexible sleeve material, for example a fluoropolymer, such as expanded polytetrafluoroethylene. Other coverings may be used, such as urethane and silicone. In one embodiment, the material of the unsupported flexible sleeve 202 may be used to form the atraumatic covering around the helical coil wrap 230.

The diameter of the wave anchor 2810 may be between about 40 mm and about 60 mm, in order to provide a seal and to avoid trauma to the intestine. For example, the diameter may be between about 50 mm and about 60 mm. In one example, the wave anchor 2810 has a diameter of about 44 mm; in another, about 56 mm. The wave anchor 2810 may comprise a length of between about 25 mm and about 60 mm. The wave anchor 2810 may comprise a wire of a diameter of between about 0.016 inches and about 0.040 inches, such as a diameter of between about 0.032 inches and about 0.035 inches.

The helical coil wrap 230 may be wrapped around the cycles of the wave anchor 2810, such around all five, six or seven wave cycles of the wave anchor 2810.

FIGS. 4A-4D are views of a portion of a distal element in an anchor in accordance with an embodiment of the invention. In particular, FIG. 4A is a side view showing a portion of the wave anchor 2810 and helical coil wrap 230 wrapped around it; FIG. 4B is a side view showing the wave anchor 2810 with helical coil wrap 230; FIG. 4C is a top view of the wave anchor 2810 with helical coil wrap 230; and FIG. 4D is a trimetric view of the wave anchor 2810 with helical coil wrap 230. It can be seen that helical coil wrap 230 is wrapped around the cycles of the wave anchor 2810, here having five crowns, although different numbers may used. The helical coil wrap 230 extends substantially along the same route as the wire that forms the wave anchor, i.e., being helically wrapped around the path taken by the wire that forms the wave anchor 2810.

Returning to FIG. 2, in accordance with an embodiment of the invention, the planar proximal element 220 prevents distal migration only by being large enough that it cannot fit through the pylorus 108. In addition, a lack of leading edges inherent in this geometry makes it hard for the proximal element to push through the pylorus. There is no need for the proximal element 220 to form a seal to the stomach wall, nor for it to penetrate tissue of the stomach; and indeed, forces against the tissue required for sealing and members that penetrate tissue are undesirable in the proximal element 220 because the antrum of the stomach is a very active region that frequently undergoes contractions. The proximal element 220 can move relatively freely within the stomach (subject to the tether and contact with food and the walls of the stomach) without engaging tissue or forming a seal. When planar, in one embodiment, the proximal element 220 is atraumatic to the stomach tissue, since it orients in the plane of the stomach when the stomach is relaxed and without food and therefore typically the stomach is flat in this state. Likewise, the proximal element 220 should have as little mass as possible, in order to avoid trauma. The planar proximal element 220 may, for example, be a planar hoop or ring with an empty middle, as shown in FIG. 2. The hoop or ring may have a diameter between about 40 mm and about 100 mm. Using a diameter too small may risk the proximal element 220 migrating distally through the pylorus into the intestine; whereas using a diameter that is too large may risk producing trauma to the stomach. In one example, the proximal element may be a ring of about 60 mm diameter. The proximal element 220 should also be easy to deliver, which may be performed by bending the ring to fit within a container that can fit through the mouth and esophagus. The proximal element 220 is formed of a resilient material that can be deformed and return to its original shape. For example, the proximal element 220 may be formed of a metal such as stainless steel or Nitinol, or a polymer, such as polyethylene, polytetrafluoroethylene, polypropylene or silicone. The proximal element 220 may also be braided, such as a braided metal or polymer. In one example, the proximal element 220 is a ring of about 60 mm in diameter, formed of nitinol or stainless steel, coated with silicone or urethane. This could be a polymer coating or a tube that covers the element. A ring 220 may be formed by joining together nitinol components using one or more crimps 228 or by welding, and may then be covered with an atraumatic substance.

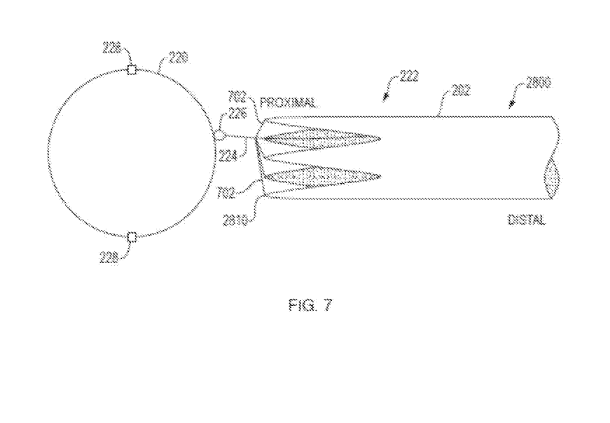

In accordance with an embodiment of the invention, the proximal element 220 may be normally oriented perpendicular to the lumen of the pyloric sphincter or the intestinal lumen, as shown in FIG. 5. More specifically, in normal use of the implant device, a central longitudinal axis of the planar proximal element, perpendicular to a plane in which the planar proximal element lies, is substantially perpendicular to a central longitudinal axis of a lumen of pyloric sphincter of the stomach, or of the intestine. Because the proximal element 220 moves relatively freely, subject to tension from the tether and contact with chyme and the stomach walls, it will also be oriented at a variety of different angles in use.

While the planar proximal element 220 is described in certain embodiments as “planar” it has some thickness in practice. The planar proximal element is significantly larger than a diameter of a pylorus of the stomach in a first dimension, and is smaller than the diameter of the pylorus in a second dimension, orthogonal to the first dimension. For example, the planar proximal element may be between about 40 mm and about 100 mm in size in the first dimension (e.g., diameter); such as between about 50 mm and about 100 mm in size in the first dimension (e.g., diameter); and between about 0.5 mm and about 15 mm in size in the second dimension (e.g., thickness and bending), such as between about 1 mm and about 5 mm in size in the second dimension (e.g., thickness). In one example, the planar proximal element comprises a hoop of between about 40 mm and about 70 mm diameter and of between about 1 mm and about 5 mm in thickness. The planar proximal element may comprise other planar shapes in addition to a ring or hoop, such as a polygon or an ellipsoid shape.

In accordance with an embodiment of the invention, the distal element 222 has the purpose of both sealing to the tissue of the intestines, and preventing proximal migration. By forming a seal, the distal element 222 permits chyme to be channeled into the distal element without contacting the walls of the intestines, thereby forming an intestinal bypass. The distal element 222 is a wave anchor 2810 (shown in FIG. 2), to which an unsupported, flexible sleeve 202 may be attached. The sleeve may be floppy, flexible, conformable and collapsible. For example, the sleeve may be one taught in U.S. Pat. No. 7,981,163 B1, the entire disclosure of which is hereby incorporated herein by reference, or any of the previously cited U.S. patents. The distal element 222 may also comprise another three dimensional object, such as a balloon and/or a toroidal element, and may include a fluid-filled chamber. For example, the distal element may comprise a fluid-filled toroidal element such as those taught in U.S. Patent App. No. 2011/0004228 A1 of Priplata et al., the entire disclosure of which is hereby incorporated herein by reference. The distal element 222 may support a restrictive element, such as a plate resistor, which may be combined with the wave anchor 2810 a sleeve; or may be used with the wave anchor 2810 without a sleeve. The resistor extends across the wave anchor and/or a sleeve and has one or more restrictive apertures therein. Further, the distal element 222 may support a catheter or a diagnostic device, such as a pressure sensor. In length, the distal element 222 may be of about the same length as the duodenal bulb 119, in order to fit within that anatomical feature. The wave anchor may comprise a single wave of a few cycles, five being shown. The wave may be formed of collapsible wire, such as of metal such as Nitinol. The distal element 222 may include no tissue penetrating features, such as barbs, and may be coated or covered with an atraumatic substance such as silicone or urethane.

In accordance with an embodiment of the invention, the purpose of the tether 224 is to couple the proximal element 220 to the distal element 222. Using a single tether 224 provides the advantage of avoiding tangling, as could occur if multiple tethers were to be used. The tether 224 may be flexible such as a suture or may be rigid, such as a rod. Regardless of whether flexible or rigid, the tether 224 may be attached at its proximal end to a ring 226, which permits the proximal element 220 to rotate independently of the tether 224 by sliding through ring 226. The tether 224 is between about 10 mm and about 50 mm in length. If the tether is too short, it may force the pylorus open, causing discomfort, but if it is too long, it may permit the distal element 222 to move too far into the intestine, such as out of the duodenal bulb. The tether has a diameter of between about 0.5 mm and about 5 mm, such as between about 1 mm and about 2 mm. A diameter that is too large may cause the pylorus to sense the tether. If too small, it could cause cutting of the tissues. In one example, the tether is a suture. The tether may be attached at its distal end to the center of a set of spokes, such as spokes made of sutures, which extend to the inner periphery of the distal element 222. The tether may, for example, be formed of polypropylene braid, or polyethylene or ptfe; and may be either uncovered or covered, for example with silicone, ePTFE or urethane to prevent trauma.

In another embodiment, the tether 224 may be attached at its distal end to the distal element 222 in a way that causes the distal element 222 to change its shape. For example, the tether 224 may be coupled to a distal crown of the wave anchor 2810, which tends to cause the wave anchor to open radially outwards at its proximal end when the tether 224 exerts tension on the distal element 222. In this way, the distal element 222 actively resists proximal migration. Other active elements may be used for the distal element 222.

In the embodiment of FIG. 2, the gastrointestinal implant device 2800 includes a sleeve 202 and an anchoring device 2810 for anchoring the gastrointestinal implant 2800 device in the duodenum 104. The anchoring device 2800 includes the wave anchor 2810 coupled to a proximal portion of the sleeve 202. The wave anchor 2810 includes a compliant, radial spring shaped into an annular wave pattern about a central axis, providing an outward radial force, while allowing substantial flexure about its perimeter. Such flexure is advantageous as it may be collapsed radially to allow for minimally-invasive delivery and ensures that the device will substantially conform to the surrounding anatomical structure when implanted and allowed to expand. The annular wave element can be formed from one or more elongated resilient members and defines a lumen along its central axis formed between two open ends.

When implanted, as shown in FIG. 5, the proximal element 220 moves substantially freely in the stomach, while the central axis of the distal element's anchor 2810 is substantially aligned with the central axis of the duodenum 104, allowing chyme to pass through the device 2800. (Note that the helical coil wrap 230 is not shown in FIG. 5 for the sake of clarity). Additionally, the compliant wave anchor 2810 minimizes trauma to the tissue by providing sufficient flexibility and compliance, while minimizing the likelihood of tissue erosion—and in particular minimizes trauma and potentially other drawbacks by including helical coil wrap 230.

The compliant wave anchor 2810 can be manufactured from a resilient metal such as a heat-treated spring steel, stainless steel, or from an alloy such as NiTi alloy commonly referred to as Nitinol. Other alloys include nickel-cobalt-chromium-molybdenum alloys possessing a unique combination of ultrahigh tensile strength, such as MP35N. Additionally, the wave anchor 2810 can be formed from a polymer and/or a composite having similar properties. The wave anchor 2810 can be manufactured from a single strand, such as a wire, contoured into the desired shape. Alternatively, the wave anchor 2810 can be manufactured from multi-strands of the same or different materials similarly contoured to the desired shape. In some embodiments, the wave anchor 2810 can be cut into the wave shape from tubular stock of the desired material, such as Nitinol.

When implanted, the anchor 2810 can enable a sleeve 202, or barrier to be securely implanted within the duodenum 104, preferably providing a fluid seal at the proximal end. To enhance a fluid seal, the proximal end of the sleeve can be contoured to the wave anchor as shown in FIG. 2. For a device 2800 using a sleeve 202 contoured to the wave anchor 2810, the proximal end appears tulip-shaped.

In an embodiment according to the invention, the proximal element 220 may prevent the device from migrating distally, but may be without a seal, whereas the distal element 222 is used to form a seal against tissue, such as the duodenal wall.

In one example, the wave anchor 2810 has five peaks, formed from wire of 0.032-0.035 inches. The hoop 220 may, for example, be 0.034 inches thick, formed of two loops with two crimps; or 0.025 inches thick, formed of three loops with two crimps.

A wave anchor as large as 55 mm or 60 mm diameter may also be used, with no tissue engaging barbs. It will be appreciated that a variety of different diameters, wire thicknesses, compliances and number of crowns for a wave anchor may be used (for example, five or six crowns on the wave anchor).

FIG. 6 shows an embodiment in which the wave anchor 2810 is tethered with two tethers.

FIG. 7 shows an embodiment in which the tether is coupled to spokes 702 on the distal element. Note that the helical coil wrap 230 is not shown in FIGS. 6 and 7 for the sake of clarity.

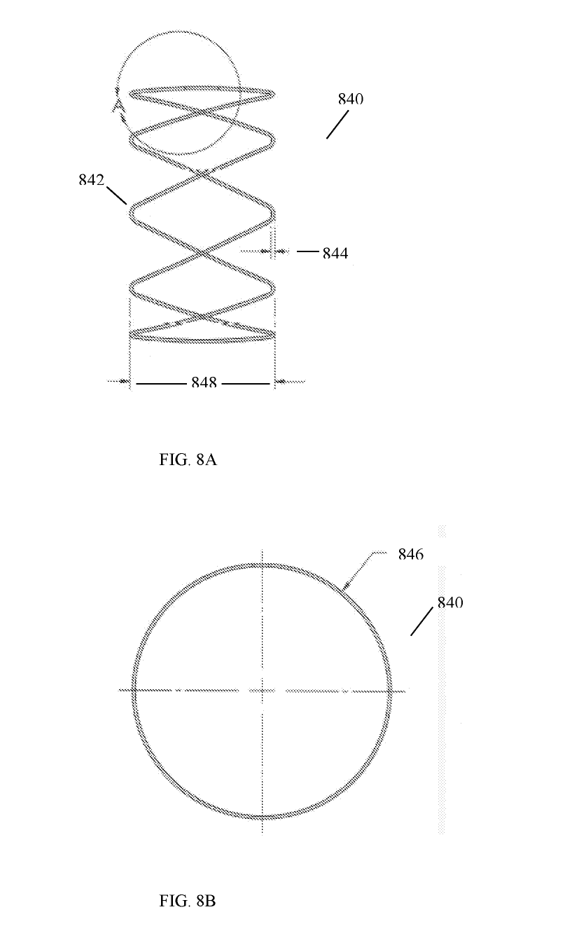

FIG. 8A is a side view, and FIG. 8B is a top view, of a wire form 840 used as the basis of a wave anchor for a distal element of a gastrointestinal implant device, in accordance with an embodiment of the invention. As shown in FIG. 8A, the wire form 840 has five crowns 842 on each side (proximal and distal crowns), although different numbers of crowns may be used. The wire form 840 can have a wire diameter 844 of between about 0.032 inches and about 0.035 inches, and may be, for example, a Nitinol wire of a diameter 844 of about 0.032 inches, although other materials and diameters may be used. The wire form 840 can comprise a cross-anchor diameter 846 of between about 50 mm and about 60 mm, such as about 56 mm. The wire form 840 can comprise a length 848 of between about 25 mm and about 60 mm, for example about 30 mm.

As used herein, it should be appreciated that dimensions given for diameters of wave anchors are exclusive of additional material that is added to the wire form in order to produce the wave anchor. For example, the diameter of any helical coil wrap is not included in the dimensions given herein for the wave anchor diameters. Thus, for example, wave anchor diameters given are the same as those given here for wire form 840, such as between about 50 mm and about 60 mm.

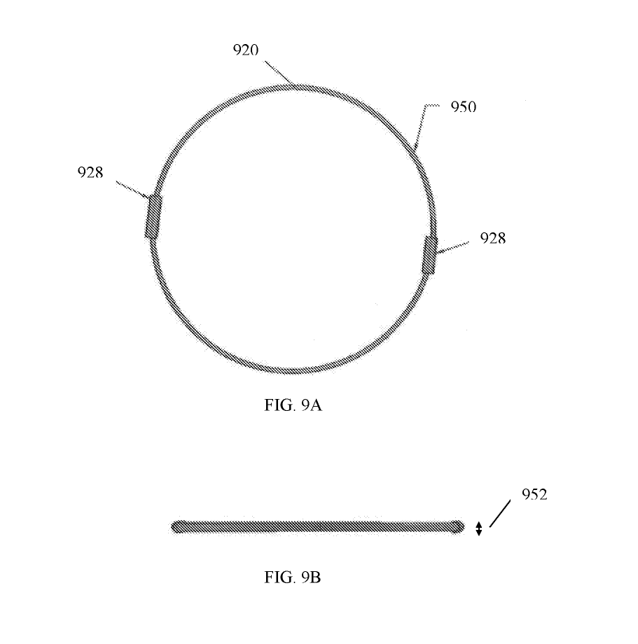

FIG. 9A is a top view, and FIG. 9B is a side view, of a hoop 920 that can be used for the proximal element of a gastrointestinal implant device, in accordance with an embodiment of the invention. The hoop 920 is of between about 40 mm and about 70 mm in diameter 950 across its opening, such as about 60 mm diameter, and of between about 0.5 mm and about 5 mm in thickness 952, such as by having a wire thickness 952 of between about 0.032 inches and about 0.036 inches, for example about 0.034 inches. One or more crimps 928 can be used to hold together the hoop.

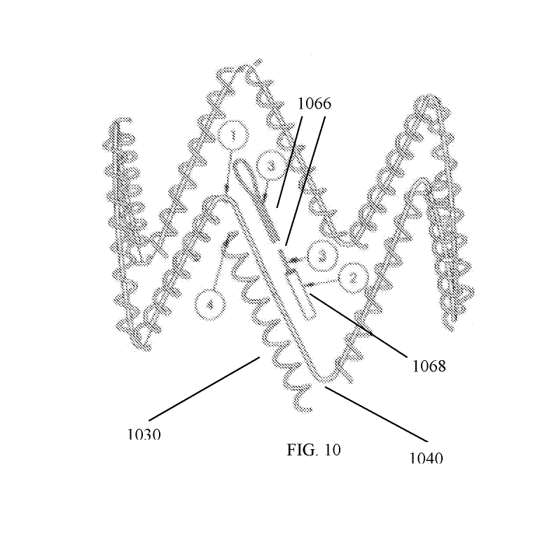

FIG. 10 is an exploded view of helical coil wraps 1030 being assembled onto the wave anchor wire form 1040 as part of assembling a distal element, in accordance with an embodiment of the invention. The helical coil wraps 1030 can be assembled onto the wire form 1040 using filler wires 1066 and a strut crimp 1068.

The planar proximal element, distal element and tether are configured to be delivered through the mouth and esophagus into a gastrointestinal tract. For example, container delivery techniques may be used that are similar to those taught in U.S. Pat. No. 7,837,643 B2 of Levine et al., the entire teachings of which are hereby incorporated herein by reference.

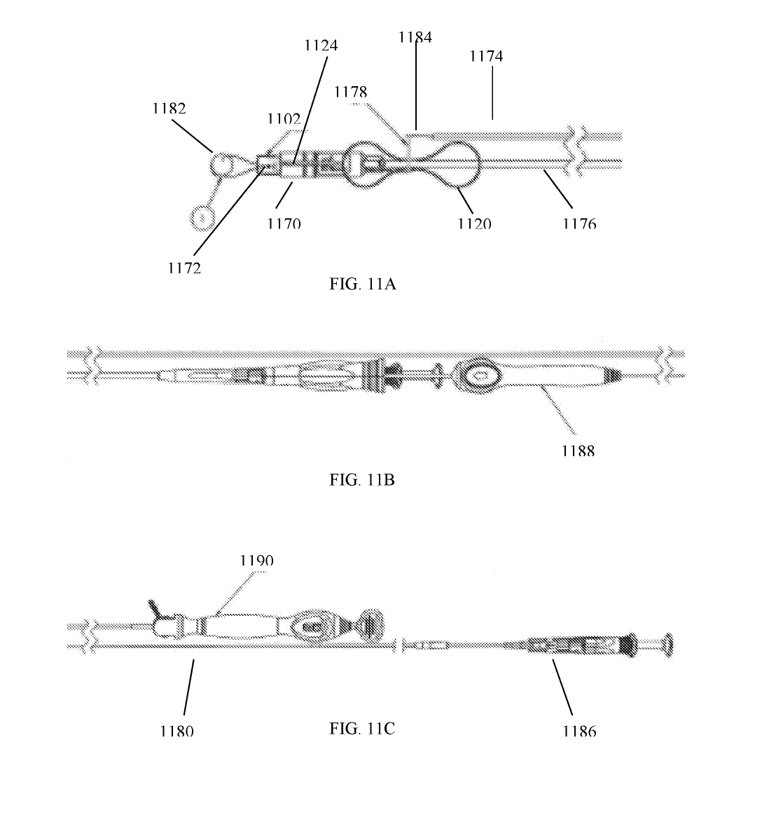

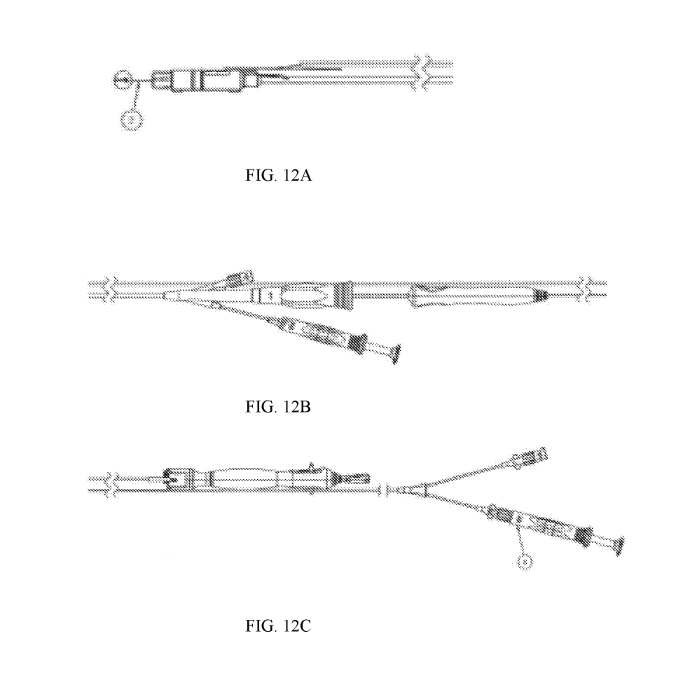

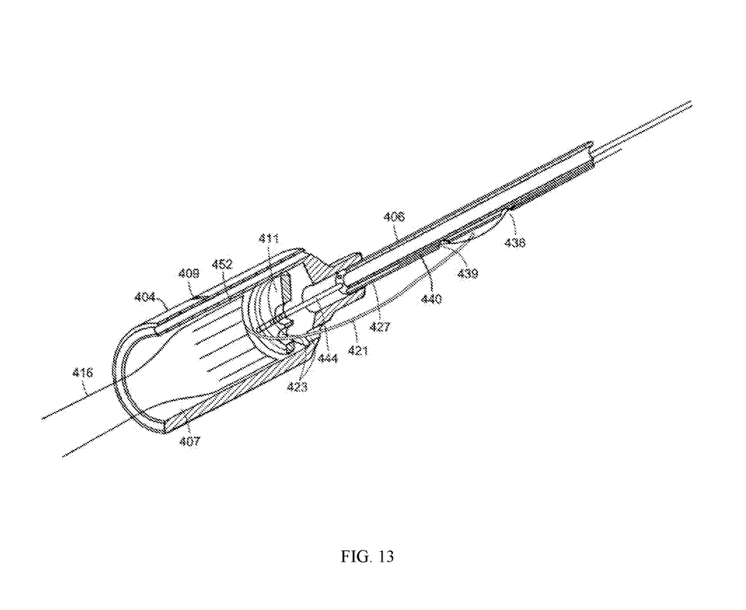

FIGS. 11A-11C are top views of a catheter-based delivery system, and FIGS. 12A-12C are side views of the same, in accordance with an embodiment of the invention. In FIGS. 11A-11C, the figures are to be viewed as a left portion view (FIG. 11A), center portion view (FIG. 11B) and right portion view (FIG. 11C) of the catheter-based delivery system, from a top view; while in FIGS. 12A-12C, the figures are to be viewed as a left portion view (FIG. 12A), center portion view (FIG. 12B) and right portion view (FIG. 12C) of the catheter-based delivery system. A container assembly 1170 stores a proximal end of the distal element, such as the wave anchor, which may be compressed into the container assembly 1170, while the distal end of the distal element, such as the sleeve 1102, may be partially folded inside the container assembly 1170 and partially extending outside of it. The container assembly comprises an opening 1172 through which the tether 1124 extends to the exterior of the container assembly 1170. A grasper catheter 1174 holds the proximal element 1120 in a collapsed state (for example, by compressing a center portion of the hoop of element 1120, as shown in FIG. 11A), exterior to the container assembly 1170, prior to release of the proximal element 1120 in the stomach. Both the delivery catheter 1176 and the grasper catheter 1174 are inserted through the mouth and esophagus into the stomach, together, with the grasper catheter 1174 holding the proximal element 1120 in the collapsed state. The grasper catheter 1174 is then used to release the proximal element 1120 from the collapsed state in the stomach, for example by releasing a suture 1178 that was being used to hold the proximal element 1120 in the collapsed state. The distal end of the delivery catheter 1176 is then extended into the intestine, such as the duodenal bulb. An inner extension, such as an inner catheter 1180, is then used to draw a portion of the sleeve 1102 from the anchor and from the container assembly 1170 as the anchor is retained container assembly 1170. The inner catheter can be releasably secured to a distal end of the sleeve, so that the sleeve can be released after the sleeve is extended. The inner catheter 1180 can also comprise an atraumatic tip 1182, such as a ball, to assist in guiding the distal end of the delivery catheter through the pylorus and into the intestine. The atraumatic tip can comprise a guidewire rail channel that runs within the interior of the atraumatic tip, to assist in guiding the tip over a guidewire. The ball may be released into the intestine after the sleeve 1102 is delivered. After the portion of the sleeve 1102 is drawn from the container assembly 1170 and released, an anchor plunger is used to displace the anchor from the container assembly, to expand into the duodenal bulb. With this, the gastrointestinal implant devices taught herein can be delivered, including, for example, a proximal element that comprises a hoop, a distal element that comprises a self-expanding wave anchor, and a tether coupling the proximal element with the distal element. By delivering in this fashion, the tether spans the pylorus, with the proximal element positioned in the stomach and the distal element positioned in the intestine. The grasper catheter may comprise a grasper hook 1184 at a distal end of the grasper catheter, and indeed the grasper catheter 1174 may be the same type of catheter that can be used for removal of the implant. The system further includes an inner catheter handle 1186, an outer catheter handle 1188, and a grasper catheter handle 1190. FIG. 13 is a cross-sectional diagram showing the internal details of a container assembly 404 with implant therein, in accordance with an embodiment of the invention. Container 404 defines storage chamber 407. Container 404 includes visual marker 409 which can be used to determine if container 404 is in a desired location before an anchor is fully expelled from container 404.

Container 404 is attached or assembled to outer catheter 406 (a portion of which is omitted from FIG. 13 for clarity). Anchor pusher wire 444 extends through an anchor pusher wire lumen which is defined by outer catheter 406. The distal end of anchor pusher wire 444 is attached or assembled to anchor pusher plate 411.

A stored portion of a gastrointestinal device includes anchor 452 and a proximal portion of sleeve 416 (the tether and proximal element are not shown in FIG. 13). Anchor 452 is collapsed or contracted and stored within chamber 407. In some embodiments, the anchor stored within the chamber(s) defined by a container assembly is a self-expanding anchor. Anchor 452 is contained or stored in container 404 during portions of a placement method that include directing the container assembly and portions of the gastrointestinal device to various locations within a gastrointestinal tract of a mammal.

Anchor retaining wire 421 extends out of the proximal end of container 404 via anchor retaining wire port 423 defined by anchor pusher plate 411 and container 404. Anchor locking wire 440 extends through anchor locking wire lumen 427 which is defined by outer catheter 406. Wire 440 emerges from lumen 427 via anchor locking wire port 438, extends through drawstring 421, and extends back into lumen 427 via anchor locking wire port 439.

After sleeve 416 has been deployed to a desired extent and container 404 is in the desired location, anchor 452 and the proximal portion of sleeve 416 can be released from container 404. Anchor locking wire 440 is pulled proximally at anchor locking wire port 438 on the proximal end of outer catheter 406, thereby pulling the distal portion of wire 440 from anchor locking wire port 439 and disengaging wire 440 from anchor retaining wire 421.

Once anchor 452 has been released from anchor locking wire 440, anchor 452 and proximal portion of sleeve 416 are expelled from container 404. To expel anchor 452 and the proximal portion of sleeve 416, a practitioner pushes anchor pusher wire 444 distally, thereby directing plate 411 and forcing anchor 452 from the distal end of container 404. In an embodiment according to the invention, the device may be removed as follows. A distal element of the gastrointestinal implant device is engaged with a removal catheter, such as the grasper catheter of FIG. 11, or such as the US Endoscopy Raptor Grasper, sold by US Endoscopy of Mentor, Ohio, U.S.A. A proximal portion of the distal element is then collapsed with the removal catheter. The distal element is moved through a pylorus of the mammal into a stomach of the mammal, with the removal catheter. The distal element is moved through an esophagus of the mammal, with the removal catheter, while towing a proximal element of the gastrointestinal implant device behind the distal element in the stomach, with the proximal element being coupled to the distal element by one or more tethers of the gastrointestinal implant device. The proximal element is moved through the esophagus with the removal catheter, at least a portion of the proximal element being collapsed by being forced against the tissue of the esophagus as the proximal element is moved through the esophagus. The distal element can be engaged by grasping a drawstring of the gastrointestinal implant device with a grasper of the removal catheter, and the proximal portion of the distal element (such as the anchor) can be radially collapsed with the drawstring. An esophageal overtube can be placed in the esophagus, the overtube forcing the proximal element to collapse as the proximal element is moved through the esophagus.

Further, the foregoing methods may be used in conjunction with removal techniques taught in U.S. Pat. No. 8,057,420, the entire teachings of which are hereby incorporated herein by reference. The proximal element and/or the distal element further may comprise a removal drawstring.

In one embodiment, a method of removing a gastrointestinal implant comprises severing one or more tethers that couples a planar proximal element of the gastrointestinal implant in a stomach with a distal element of the gastrointestinal implant in an intestine; with a catheter, removing the planar proximal element proximally out of the stomach through a mouth; and with a grasper on a distal end of the catheter, grasping a drawstring to remove the distal element proximally out of the intestine, though the stomach and out of the mouth. The planar proximal element may be removed through an overtube. The distal element may be collapsed radially into a retrieval hood. The proximal element may also be collapsed in a retrieval hood for removal.

In accordance with an embodiment of the invention, there is provided a method of treatment. The method comprises providing a gastrointestinal implant device set forth herein, and securing the device across the pylorus of a patient. Chyme may be channeled from the stomach into an unsupported, thin-walled sleeve extending into the intestine from the gastrointestinal implant device. For example, a sleeve such as those set forth in U.S. Pat. No. 7,682,330 (the entire disclosure of which is hereby incorporated herein by reference) may be used. Further, a flow of chyme from the stomach into the intestine may be restricted with a restrictor coupled to the gastrointestinal implant device. For example, a restrictor such as those set forth in U.S. Pat. No. 7,771,382 (the entire disclosure of which is hereby incorporated herein by reference) may be used. In addition, a combination of both a restrictor and a sleeve may be used with anchors taught herein.

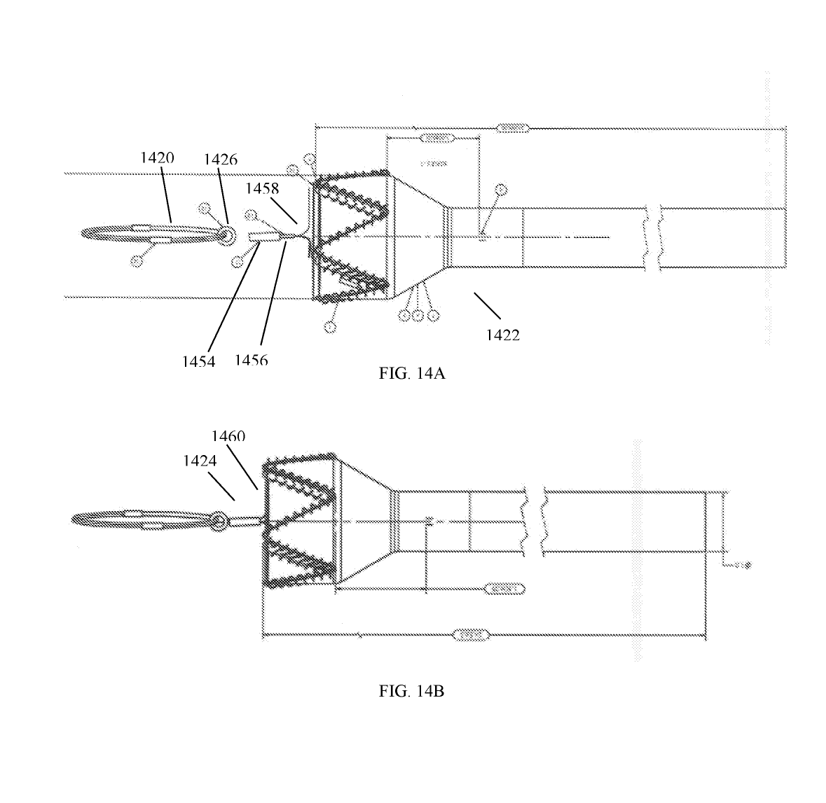

In accordance with an embodiment of the invention, a single tether, or more than one tether can couple the proximal element to the distal element. FIG. 14A is a disassembled view, and FIG. 14B is an assembled view, of a gastrointestinal implant device in accordance with an embodiment of the invention, illustrating use of a tether 1424 that includes at least one suture 1458. A tether crimp 1456 is used to hold the at least one suture 1458 together in a central region of the distal element 1422. An overtube 1454, such as a silicone overtube, is used to cover the tether crimp 1456. A ring 1426, such as a steel ring, couples the tether 1424 to the hoop 1420. One or more drawstrings 1460, such as two drawstrings, permit collapsing of the proximal end of the distal element 1422 for removal of the implant.

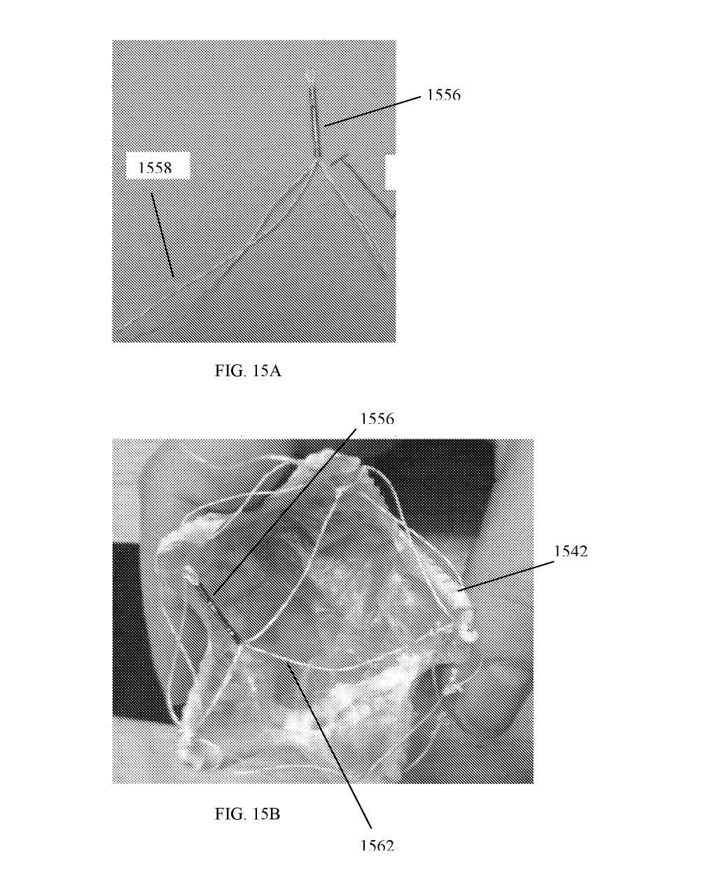

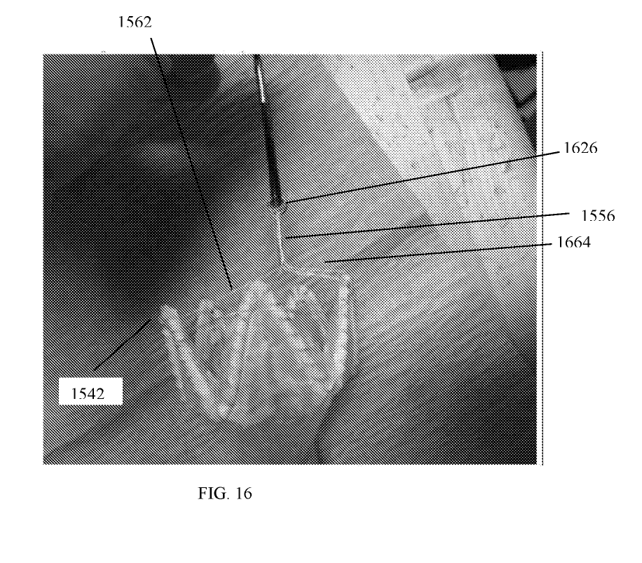

FIGS. 15A, 15B and FIG. 16 are diagrams illustrating assembly of a tether using one or more sutures, in accordance with an embodiment of the invention. FIG. 15A shows assembly of a suture with a crimp for use in a tether; FIG. 15B shows a suture structure with crimp, coupled to the crowns of a gastrointestinal implant; and FIG. 16 shows a fully assembled gastrointestinal implant using the crimped suture structure; in accordance with an embodiment of the invention. In FIG. 15A, at least one suture 1558, which may be a single suture, is folded, looped, and crimped with tether crimp 1556. In FIG. 15B, the at least one suture has been tied to a plurality of crowns 1542 of the wave anchor of the distal element, for example three crowns. Double double surgeon's knots may be used to tie the suture through openings in at least one of the crowns 1542. It can be seen in FIGS. 15B and 16 that the tether crimp 1556 is located in a center region (viewed from above the opening of the distal element, as in FIG. 15B) of the distal element, with segments 1562 of the at least one suture extending between the tether crimp 1556 and the crowns 1542. As can be seen in FIG. 16, the at least one suture is coupled not only to the crowns 1542, but also to the proximal element, by virtue of being coupled to ring 1626 which is in turn coupled to the proximal element. The at least one suture may be a single suture which is looped and folded back on itself to obtain the suture arrangement shown in FIGS. 15A, 15B and 16. In addition, a second suture may function as a “fail safe” or backup tether. For example, a second suture 1664 may be coupled to a crown of the wave anchor (such as only a single crown), run through the same overtube 1454 (see FIG. 14) as the first tether, and be coupled to the ring 1626. Other numbers of sutures may be used.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A gastrointestinal implant device comprising:
an element configured to reside in an intestine to resist proximal migration, the element comprising a wave anchor and a surface roughness element on a surface of at least a portion of the wave anchor; and
an unsupported, thin-walled sleeve coupled to the element configured to reside in the intestine.

2. The gastrointestinal implant device of claim 1, wherein the wave anchor comprises a diameter of between about 40 mm and about 60 mm.

3. The gastrointestinal implant device of claim 1, wherein the wave anchor comprises a length of between about 25 mm and about 60 mm.

4. The gastrointestinal implant device of claim 1, wherein the wave anchor comprises a wire of a diameter of between about 0.016 inches and about 0.040 inches.

5. The gastrointestinal implant device of claim 1, wherein the surface roughness element comprises a plurality of spheres on at least one crown of the wave anchor.

6. The gastrointestinal implant device of claim 1, wherein the surface roughness element comprises a helical coil wrap around at least a portion of the wave anchor.

7. The gastrointestinal implant device of claim 6, wherein the helical coil wrap comprises a wire of a diameter of between about 0.008 inches and about 0.020 inches.

8. The gastrointestinal implant device of claim 6, wherein the helical coil wrap comprises an outside diameter of between about 0.080 inches and about 0.250 inches across a helix of the helical coil wrap.

9. The gastrointestinal implant device of claim 6, wherein the helical coil wrap comprises a coil wrap spring pitch of between about 8 coils per inch and about 60 coils per inch.

10. The gastrointestinal implant device of claim 6, wherein the element configured to reside in the intestine comprises an atraumatic covering over at least a portion of the helical coil wrap.

11. The gastrointestinal implant device of claim 1, wherein the device comprises no tissue penetrating features.

12. The gastrointestinal implant device of claim 1, wherein the element configured to reside in the intestine comprises a restrictor.

13. The gastrointestinal implant device of claim 1, further comprising:
a proximal element configured to reside in a stomach to resist distal migration;
a distal element comprising the element configured to reside in the intestine to resist proximal migration; and
one or more tethers coupling the proximal element to the distal element.

14. The gastrointestinal implant device of claim 13, wherein, in normal use of the implant device, a central longitudinal axis of the proximal element, perpendicular to a plane in which the proximal element lies, is substantially perpendicular to a central longitudinal axis of a lumen of a pyloric sphincter of the stomach.

15. The gastrointestinal implant device of claim 13, wherein the proximal element is between about 40 mm and about 100 mm in size in a first dimension, and is between about 0.5 mm and about 15 mm in size in a second dimension, orthogonal to the first dimension.

16. The gastrointestinal implant device of claim 13, wherein the distal element is configured to seal to tissue of the intestine, thereby channeling chyme from the stomach into the intestine.

17. The gastrointestinal implant device of claim 13, wherein the proximal element is planar.

18. The gastrointestinal implant device of claim 17, wherein the proximal element comprises a hoop.

19. The gastrointestinal implant device of claim 13, wherein the proximal element is without a seal to the stomach.

20. The gastrointestinal implant device of claim 13, wherein the tether comprises a suture.

21. The gastrointestinal implant device of claim 20, wherein the tether comprises at least one suture, the at least one suture being coupled to at least one crown of the wave anchor of the distal element, and to the proximal element.

22. The gastrointestinal implant device of claim 21, wherein the at least one suture is coupled to a ring that is coupled to the proximal element.

23. The gastrointestinal implant device of claim 13, wherein the tether is between about 10 mm and about 50 mm in length.

24. The gastrointestinal implant device of claim 13, wherein the tether is between about 0.5 mm and about 5 mm in diameter.

25. The gastrointestinal implant device of claim 13, wherein the distal element is configured to change shape upon transmission of force to the distal element by the tether.

26. The gastrointestinal implant device of claim 13, wherein the tether is coupled to at least one crown of the wave anchor.

27. The gastrointestinal implant device of claim 13, wherein the proximal element is loosely coupled to the tether, thereby permitting the proximal element to rotate independently of the tether.

28. The gastrointestinal implant device of claim 13, wherein the proximal element is coupled to a ring to which the tether is coupled.

29. The gastrointestinal implant device of claim 13, wherein at least one of the proximal element and the distal element further comprises a removal drawstring.
